(19) United States
(12) Patent Application Publication
(10) Pub. No.: US 2009/0241483 A1
(43) Pub. Date: Oct. 1, 2009

(54) TEARABLE PACKAGES FOR PHARMACEUTICAL PRODUCT AND METHOD

(75) Inventors: Bruce D. Detwiler, Boyertown, PA (US); Richard C. Fuisz, McLean, VA (US)

Correspondence Address: HOFFMANN & BARON, LLP, 6900 JERICHO TURNPIKE, SYOSSET, NY, 11791, US

(73) Assignee: MonoSol Rx, LLC, Portage, IN (US)

(21) Appl. No.: 12/409,972

(22) Filed: Mar. 24, 2009

Related U.S. Application Data
(60) Provisional application No. 61/039,822, filed on Mar. 27, 2008.

(51) Int. Cl. A61B 19/02; B65D 83/04; B67B 7/92

(52) U.S. Cl. 53/492; 206/438; 206/532

(57) ABSTRACT

An aspect of the invention provides a tearable package for a pharmaceutical product including: (i) a first layer including a sheet of flexible material; (ii) a second layer including a sheet of flexible material; the layers being overlaid and fused together at a perimetrical sealing region, the fused layers defining a cavity therebetween for the storage of the pharmaceutical product; wherein one of the layers has a discontinuous region in the sealing region; and wherein the package may be folded through the discontinuous region to be torn such that the pharmaceutical contents may be removed.

### CROSS-REFERENCE TO RELATED APPLICATIONS

The present invention claims priority to U.S. Provisional Application No. 61/039,822, filed Mar. 27, 2008, the entire contents of which are incorporated herein by reference.

### FIELD OF INVENTION

The present invention relates to tearable package for pharmaceutical products. More particularly, the present invention relates to child resistant and elder friendly tearable pouch pharmaceutical packages.

### BACKGROUND OF RELATED TECHNOLOGY

Various packages have evolved in the field of packaging, including blister packs and notched tear open packages. These designs were not ideal for housing single dose thin film pharmaceutical technology. Also, the packages were neither child resistant nor user friendly. Though these technologies easily indicated the method of opening the package, users with limited grip or hand strength had difficulty in gripping the thin, laminated notched area or prying open blister packs. Thus, packaging was developed with a less evident perforation area, such as that disclosed by U.S. Patent Application No. 2006/0023976 to Alvater.

The Alvater packaging discloses a substantially flat packaging composed of two layers sealed to enclose a pharmaceutical dose. The packaging discloses a perforated area set inward from the outer edge of the overlay, such that a user has to fold over a portion of the packaging in order to successfully tear off a portion of the package to yield a partial opening to the pharmaceutical material within. Though the reference discloses packaging, inter alia, thin film pharmaceuticals, the reference results in several shortcomings.

First, as the perforation is through both layers of the package, a shear force applied to an area near the perforation may result in an unintended premature package opening, which may in turn result in contamination and compromised integrity of the drug dose.

Second, the tear away portion yields access to only a portion of the inner cavity. Though the packaging can be manipulated to allow the film to be pulled out, the thin film is much more likely to break when it is flexed and deformed upon exiting the package. Thus, portions of the dosage may be contaminated or lost entirely.

Third, the reference discloses that to gain access to the film, both layers of the packaging may be gripped and peeled away or apart from one another. This action requires both hands of a user to grip both sides of the packaging. As a result, as a user peels open the packaging; the user's hands are preoccupied while a majority of the film is open to the ambient environment. Thus, while the user is not yet able to retrieve the pharmaceutical product, the film is exposed and susceptible to contamination, blowing away, or falling onto the ground/floor or other contamination, destruction, or loss.

Fourth, the user must have adequate sight, grip, and dexterity to grip the individual, thin layers of the first and second layers of the packaging, peel the layers apart, and retrieve the thin film from within before it falls out. Often, users may have ailments or limitations in their grip, grasp, and dexterity that prevents such accuracy and precision in opening a single dose of a pharmaceutical product once, let alone repeated opening of a series of single doses over time, as with a prescribed usage.

### SUMMARY OF THE INVENTION

In field of packaging technology, there exists a need to efficiently and effectively package pharmaceuticals and other consumables to resist water and air contamination, protect sensitive actors (e.g. flavor oils or reactive pharmaceutical components), prolong shelf life, reduce contamination, promote ease of access to the contents of the package, and reduce the ease of access to the contents through child tampering. The present invention meets these and other needs.

The present invention provides a tearable package for a pharmaceutical product, particularly a film product, with at least two layers of flexible material that are fused or otherwise affixed together in order to define a cavity therebetween, which may in turn store and dispense a unit dose a pharmaceutical product. The tearable pharmaceutical package of the present invention secures the pharmaceutical product housed within from contaminants, maintains an inert environment to prolong shelf life of the pharmaceutical, and promotes a child resistant yet easy to open packing for users with limited grip, grasp, sight, or dexterity.

An aspect of the invention provides a tearable package for a pharmaceutical product including: (i) a first layer including a sheet of flexible plastic and or foil; (ii) a second layer including a sheet of flexible plastic and or foil; the first and second layers being overlaid and fused together at a perimetrical sealing region, the fused layers defining a cavity therebetween for the storage of the pharmaceutical product; wherein one of the layers has a discontinuous region in the fused region, and the other layer has a continuous region according to the discontinuous region of the other layer; and wherein the package may be folded on an axis through the discontinuous region, and when a tearing force is applied, the discontinuous region facilitates tear initiation along the fold axis through the layers and into the cavity of the package to allow the pharmaceutical product within the cavity to be removed.

In another aspect of this invention, the two layers are of different thicknesses.

Another aspect of the invention provides a tearable package for a pharmaceutical product, including: (i) a first layer including a sheet of flexible plastic and or foil, the first layer having a first thickness; (ii) a second layer including a sheet of flexible plastic and or foil, the second layer having a second thickness greater than the first thickness, the first and second layers being overlaid with the other and fused together at a sealing region, the sealed layers defining a cavity therebetween for the storage of the pharmaceutical product; and wherein the package may be folded on an axis through the sealing region, such that when a tearing force is applied, the different thicknesses facilitates tear initiation along the fold axis through the first layer and into the cavity of the package to allow the contents of the cavity to be removed.

In another aspect of the invention, one of the first and second layers may have a discontinuous region in the sealing region to promote and effectuate a tear initiation and propagation site along the fold axis.

Yet another aspect of the invention provides a method for opening a package containing a pharmaceutical product, the method including: a) providing a tearable package for a pharmaceutical product including: a first layer including a sheet of flexible material; (ii) a second layer including a sheet of flexible material; the first and second layers being overlaid and fused together at a sealing region, the fused layers defining a cavity therebetween for the storage of the pharmaceutical product; wherein the layers together provide a tear promoting feature; b) folding one edge of the package on an axis that crosses the discontinuous region and applying a tearing force to initiate a tear at the site of the discontinuous region along the fold axis along the entire side of the layers; and c) propagating the tear through at least one of the layers through the length of the packaging to allow the contents of the cavity to be removed.

These and other features will be better understood through a study of the following descriptive and accompanying exemplary drawings and claims.

### BRIEF DESCRIPTION OF THE DRAWINGS

FIG. 1 depicts an exploded perspective view of an embodiment of the present invention.

FIG. 2 depicts a top plan view of an embodiment of the present invention with an embodiment of the discontinuous region.

FIG. 3 depicts a cut-away side view across the 2-2 axis of FIG. 2.

FIG. 4 depicts an embodiment of the present invention in a folded position, prior to tearing.

FIG. 5 depicts an embodiment of the present invention torn so as to be in a partially opened state.

FIG. 6 depicts a perspective top view of an embodiment of the present invention.

FIG. 7 depicts a side plan view of another embodiment of the present invention.

FIG. 8 depicts a partially folded perspective view of another embodiment of the present invention.

FIG. 9 depicts a perspective opened view of another embodiment of the present invention.

FIG. 10 depicts a flowchart of an embodiment of the method of the present invention.

### DETAILED DESCRIPTION OF THE INVENTION

As the technology related to thin films has developed to include pharmaceutical delivery systems, the desire remains to securely store the pharmaceutical from children while employing an accessible packaging for users with limited hand strength, grip, or dexterity. The complex and reactive ingredients of pharmaceuticals requires packaging with specific resistance against contamination, reaction, or degradation of the compound so as to promote a longer a shelf-life.

Single-dose packaging has been developed in order to reduce contamination or accidental overdose with respect to thin film pharmaceuticals. The various embodiments of the present invention provide new and useful applications in the field of packaging technology, in which the various embodiments efficiently and effectively overcome the shortcomings of previous technology of package film pharmaceuticals and/or the like while resisting contamination and maintaining the integrity of the composition and its sensitive actors.

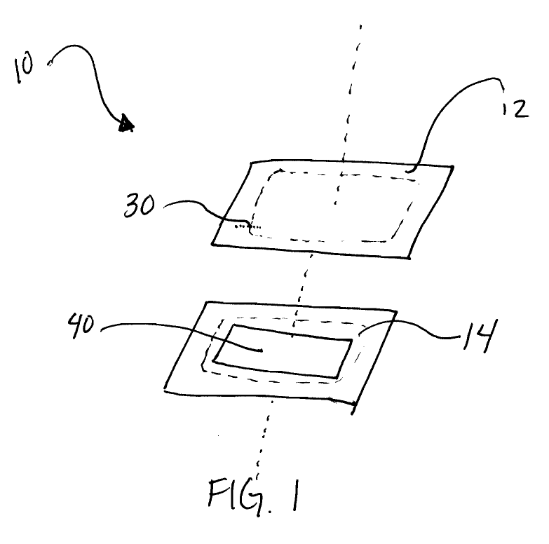

The present invention provides embodiments of a tearable package for a pharmaceutical product. The tearable package 10 is shown in, for example, FIGS. 1-9. Also, the present invention provides an embodiment of a method for opening a tearable package containing a pharmaceutical product, which is depicted in FIG. 10.

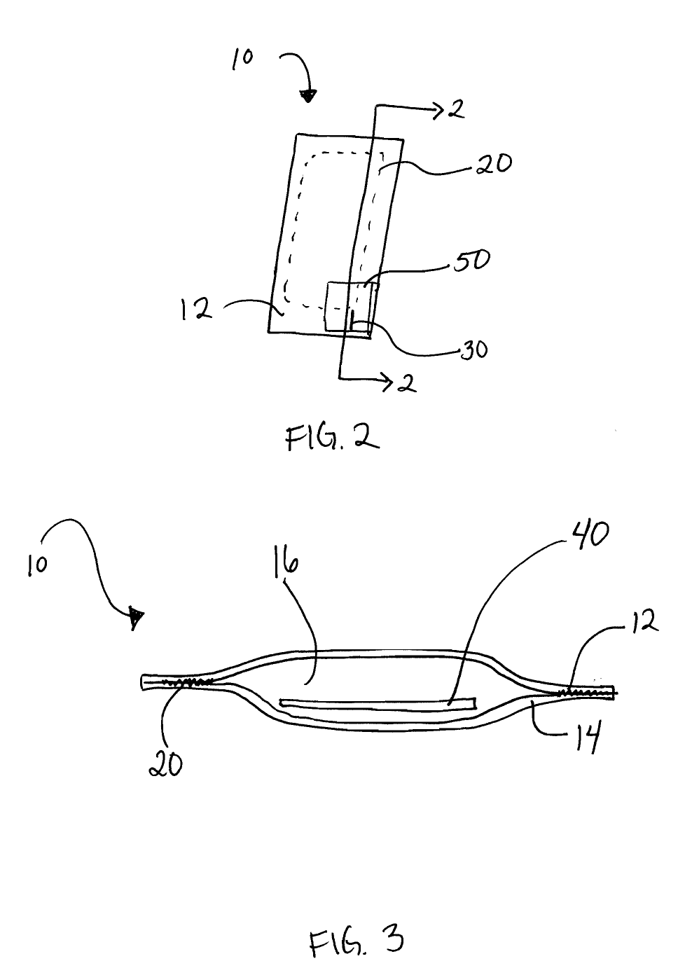

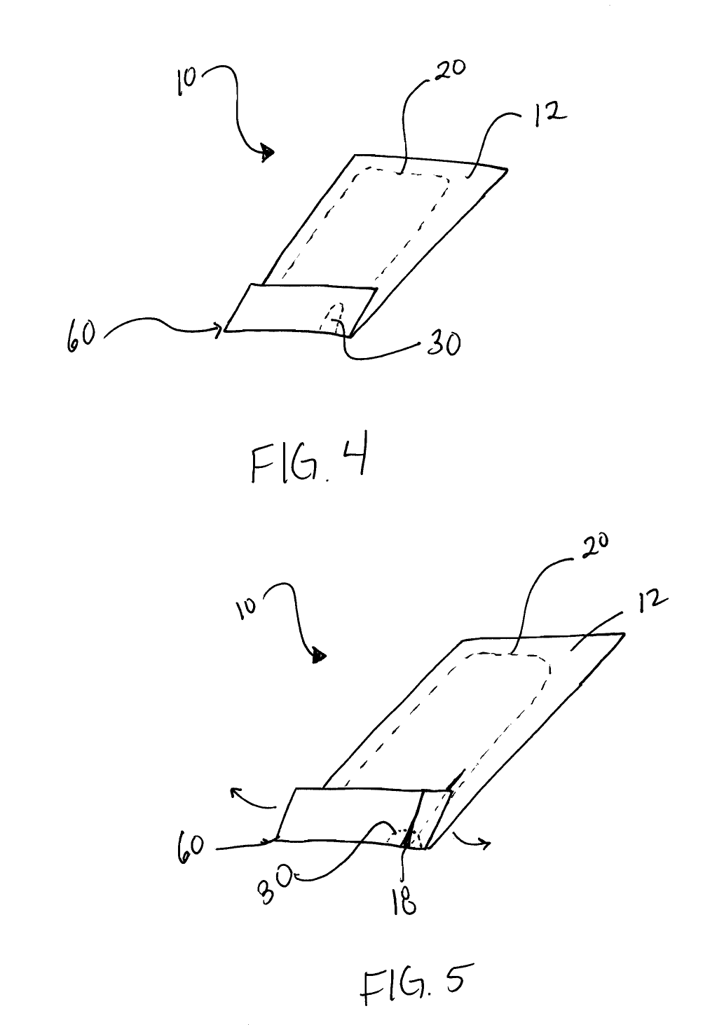

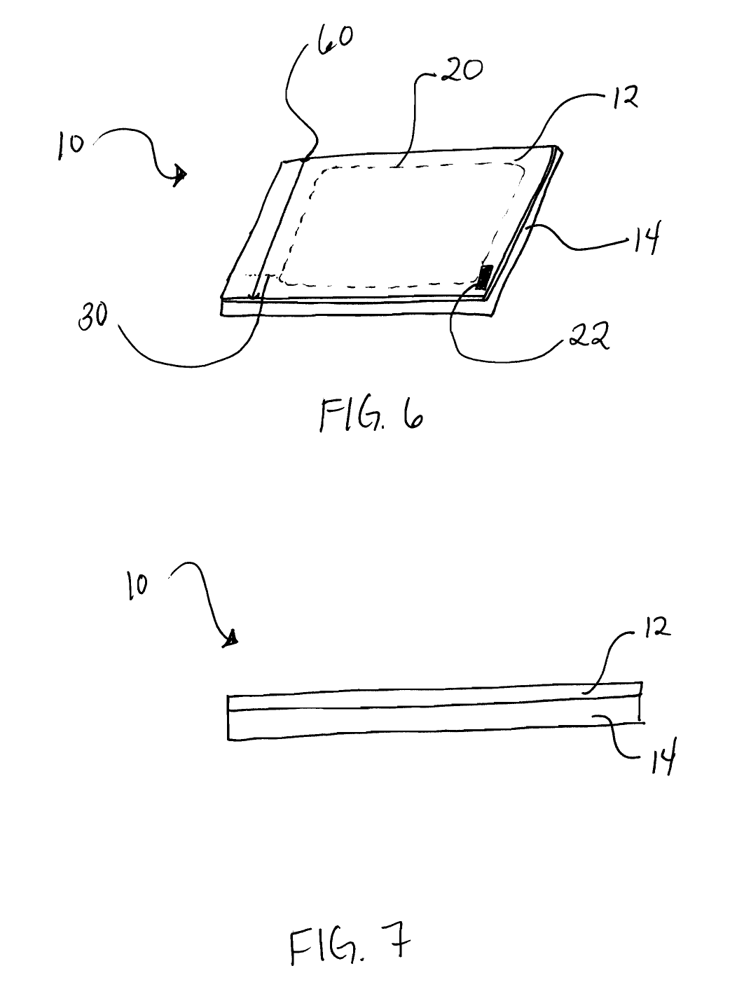

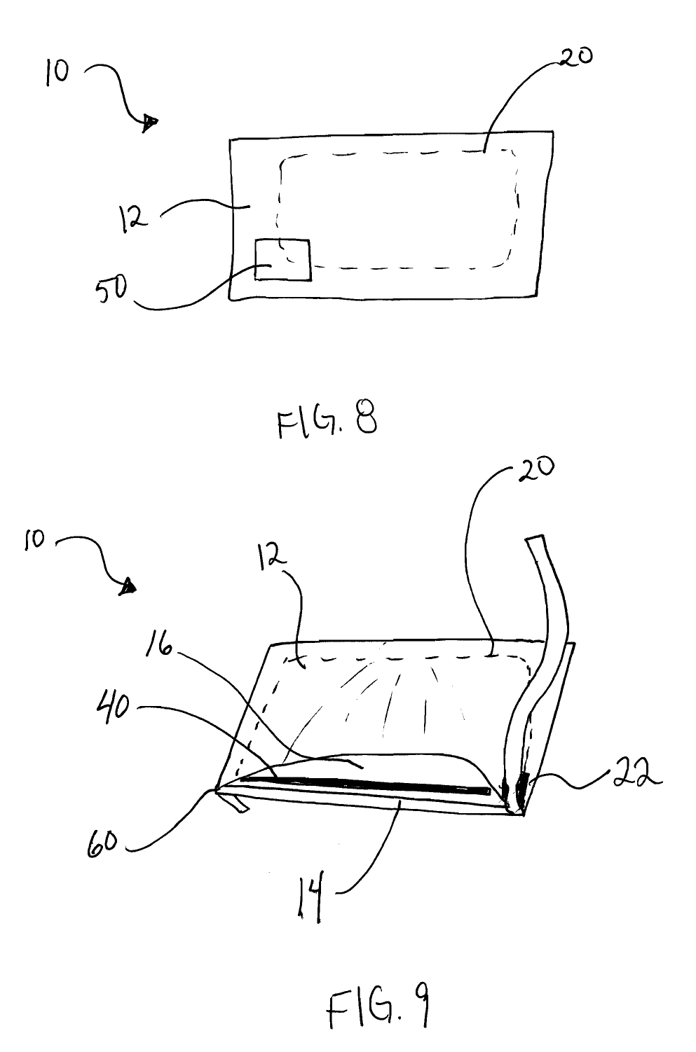

An embodiment of the tearable package 10 for a pharmaceutical product includes: a first layer 12 and a second layer 14. The first and second layers (12, 14) may be composed of a flexible plastic, a foil, a polymer, combinations thereof, and the like, as is known in the art. The first and second layers (12, 14) may be overlaid upon one another and fused together to create a sealing region 20. Referring to FIG. 1, FIG. 2, FIG. 4, and FIG. 5, one of the first and second layers (12, 14) has a discontinuous region 30. The discontinuous region 30 is located in the sealed region 20. For example, as shown in FIG. 1, FIG. 2, FIG. 4, and FIG. 5, the first sheet 12 may be configured with the discontinuous region. In the area of overlay, and the other layer (the second layer in FIGS. 1, 2, 4, and 5) has a continuous region corresponding to the discontinuous region 30 of the other layer.

Optionally, the first and second layer (12, 14) may be of the same or of different thicknesses, so as to accommodate and or facilitate a tear region.

In another embodiment of the packaging, the first layer 12 thickness is less than the second layer 14 thickness. This may be depicted, for example, in FIGS. 7-9. Thus, once the tear force is initiated, the tear transgresses along the first sheet 12, while the second layer 14 remains intact. As such, once the tear is complete, the user may compress the ends of the packaging together in order to create a puckering of the first and second layers (12, 14), thereby opening the package 10. Thus, once a user reaches for the pharmaceutical product 40, the second layer 14, which is intact, supports the pharmaceutical product 40 while it is being extracted, allowing the user to get a good grip on the product, or alternatively, sliding the pharmaceutical product 40 directly into their mouth.

Optionally, one of the first and second layers (12, 14) may have a discontinuous region 30 in order to promote and support a tear initiation in the package 10.

The sealed region 20 may be, for example, perimetrical seal around the outer edges of the layers 12, 14. As another example, a portion of the overlaid first and second layers (12, 14) inset from the outer edge may be fused to create the fusing region 20. Sealing may refer to contaminant free seals, including bonding, fusing, adhering, melting, and combinations thereof. Upon sealing, the first and second layers (12, 14) cooperate to form and define a cavity 16 therebetween. As is shown in FIG. 3, the cavity 16 is the site for storage of a pharmaceutical product 40, such as a film, pill, capsule, or the like. Similarly, the product stored within the package may be a nutraceutical or cosmetic product in the form of a pill, capsule, ointment, cream, lotion, or the like.

The discontinuous region 30 may be of one or more forms, as is known in the art. The discontinuous region 30 may include, for example, a cut, puncture, slit, or perforation through the one of the layers. The discontinuous region 30 may be in one or more shapes or designations, in order to facilitate and promote a tear initiation site. Also, the discontinuous region 30 may be in one or more shapes or patterns, as may be desired, including, for example, a generally geometric configuration, a line, or any other type of polygon type form. As examples, the discontinuous region may be an oval; a cross, a square, a rectangle, an arrow, or the like. The discontinuous region 30 may comprise one or more cuts and/or punctures in close proximity, in order to create an area of physical weakness. This area may be only a fraction of the surface area of the sheet, for example, about a few millimeters.

In order to promote a child resistant design, the discontinuous region 30 may be located on a surface of one of the two layers (12, 14), not extending to the end or edge of the layers. In such a matter, the user must be of a comprehension level to understand that the discontinuous region 30 must be properly manipulated in order to tear the package 10 and access the pharmaceutical product from within the cavity 16.

For a user to access the pharmaceutical product 40 housed in the cavity 16 of the package 10 of the present invention, the user must fold or otherwise bed a portion of the packaging over itself along the discontinuous region 30. Optionally, the embodiments disclosed may include an indent, a marking, or a pre-folded area in order to direct the user to manipulate the package 10 along the desired location to promote a tear originating at the discontinuous region 30 and extending along the tear axis to facilitate a full and proper tear on the first attempt.

At the discontinuous region 30, a pre-fold 60 may be configured on a portion of the pharmaceutical package 10 such that the axis of the pre-fold 60 intersects at least a portion of the discontinuous region 30 of one of the two layers (12, 14). The pre-fold 60 may then configure the package 10 along the discontinuous region 30, in order to align the layers (12, 14) with the tear origination site. As such, once the package 10 is folded, it may be opened so that the pharmaceutical product 40 may be accessed and retrieved from the cavity 16.

The discontinuous region 30 creates a weakening in the integrity of the package when the package is folded over along an axis through the discontinuous region. the axis, as generally referred to herein, may include the general line which corresponds to an extrapolation of the discontinuous region 30. Thus, when the package 10 may be folded along the discontinuous region 30, the tear ultimately conforms to or otherwise generally along the axis. After the tear is initiated, it may be propagated through one or both layers (12,14), as may be desired, in order to reveal or otherwise expose the cavity 16 and allow access to and removal of a pharmaceutical product 40 from the package 10.

When a tearing force is applied to the fold 60 at the discontinuous region 30, a tear 18 may be initiated and propagated along the axis of the fold 60. As such, the discontinuous region 30 may facilitate initiation of the tear 18. Once the tear 18 may transgress along the length of the package layers (12, 14), the cavity 16, is effectively bisected such that the pharmaceutical product 40 retained in the cavity 16 may be accessed and removed therefrom.

The first and second layers (12, 14) may be composed or constructed of various materials, including, for example, polymer, plastic, fabric, foil, metal, and combinations thereof. The layers (12, 14) may be constructed of tearable plastic sheeting material, for example, polythethylene terephthalate (PET), of between 25 and 100 gauge thickness. In measurements of film thickness, 1 gauge is 0.01 mil or 0.254 microns thick. The layers may be of the same or different thicknesses. Further, the thicknesses may be selected in order to promote a desired level of flexibility and/or rigidity to allow the package 10 to deform along certain constraints, while resisting forces which may damage or destroy the pharmaceutical product therein. For example, where the package 10 has a discontinuous region 30 in one of the layers (12, 14), it may be desirable to have the layer with the discontinuous region 30 be of a lower thickness than the continuous layer.

The layers (12, 14) may be affixed or fused to one another, for example, in a parametric fashion, perimetrically rounded along the edges of the overlaid layers, or along any pattern or portion of the overlaid region, such that the inner cavity 16 is created and maintained therebetween. The sealed region 20 need not have a uniform width around the perimeter of the package 10. For example, a portion of the sealed region 20 along one edge may be somewhat wider than along the other edges, so as to accommodate the discontinuous region 30. The discontinuous region 30 may be associated to a portion of the sealed region as, for example, along one side of the package 10.

Optionally, one or both of the layers (12, 14) of the package 10 may include some sort of indicia 50 or marking to indicate to a user information relevant to the package 10 or pharmaceutical product 40 housed within the cavity 16. For example, the indicia 50 may contain information indicating the discontinuous region 30, the fold line 60, or the axis along which the tear 18 may be propagated or transgressed. Alternatively, the indicia 50 may contain dosage instructions, ingredient listings, trademark information, and/or other relevant information as may be desired. The indicia 50 may be preprinted, embossed, or integral with one or more layers (12, 14). Or, the indicia 50 may be printed upon, embossed, or etched onto a surface of the package 10. The indicia 50 may include: pictorial representations, printing, or other markings which may indicate to a user a desired message or direction.

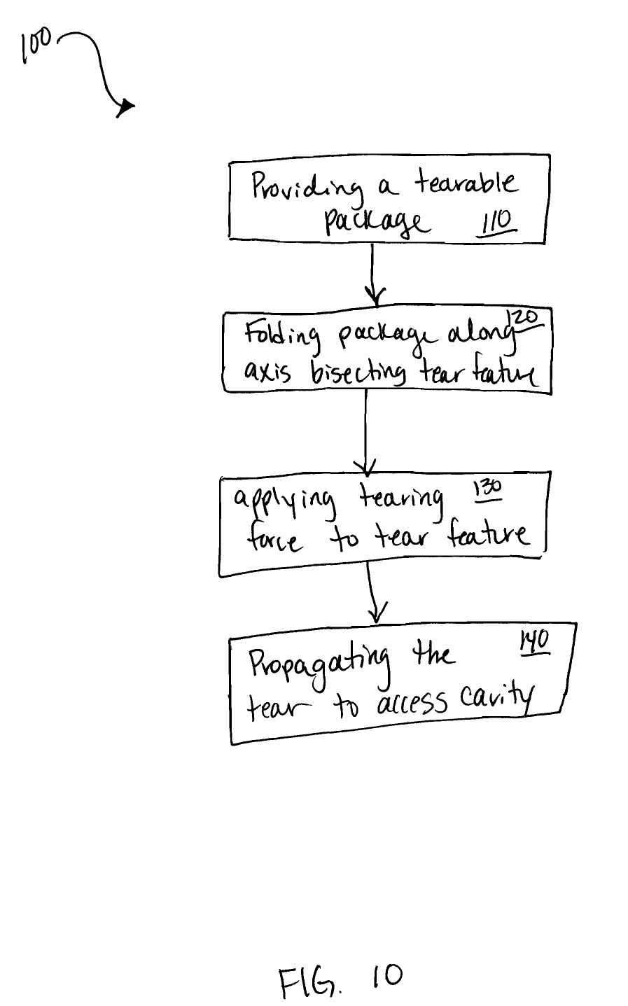

Optionally, the tearable package 10 may include an anchor 22. The anchor 22 may be located opposite the discontinuous region 30 on the other side of the cavity 16 from the discontinuous region 30. Thus, once a tear 18 transgresses from the discontinuous region 30 through at least one of the layers (12, 14) towards the end of the package 10, the anchor prevents the torn away portion from completely removing from the package 10. The anchor 22 may be shown, for example, in FIGS. 6 and 9. Thus, the anchor 22 may be employed to maintain an integral packaging, which may increase efficiency of dosing while decreasing cleanup and litter of the torn portion. Also, as such, the anchor 22 allows a user to focus on accessing and retrieving the pharmaceutical product 40 housed in the cavity 16 and not on the various parts of the package 10.

Another embodiment of the present invention includes, for example, a method for opening a packaging containing a pharmaceutical product, as is shown in FIG. 10. A method 100 for opening a package containing a pharmaceutical product, may include, for example: a) providing 110 a tearable package for a pharmaceutical product; b) folding 120 one edge of the package on an axis that crosses the tear promoting feature; c) applying 130 a tearing force to initiate a tear at the site of the tear promoting feature along the fold axis; and d) propagating 140 the tear through the layer defining the cavity to allow the contents of the cavity to be removed.

The providing step 110 may include, for example, providing one or more of the various previously described package 10 embodiments. The folding step 120 may include, for example, bending, deforming, folding, the packaging, or utilizing a pre-fold or indent as previously discussed to manipulate the layers (12, 14) appropriately along a tear feature. The tear promoting feature may include, for example, a discontinuous region, a lower thickness layer, or both, and the like. The tearing step 130 may include gripping or grasping both sides of the packaging from the tear promoting feature, followed by imposing a tear force on the package 10 sufficient to create and promote a tear along the length of the package 10.

The present invention is suited for any pharmaceutical product 40 which may be single-dose distributed, including, for example, thin film pharmaceutical, pills, capsules, lozenges, liquids, moistened towels and pre-impregnated wipes. Also, the package 10 provides a secure, contaminant free, environmentally controlled packaging. The package 10 employs elements and features that are particularly suited for allowing users with limitations to easily access and acquire the pharmaceuticals housed within, while at the same time promoting child resistant features to prevent unauthorized and/or unintended access.

In order to access the package 10, a user may employ a force sufficient or exceeding the pre-required threshold of force to tear the package 10, either along a layer of low thickness, along a discontinuous layer 30, or both.

The feature of a discontinuous region 30 in the package 10 may result in a lower threshold of tear strength required to initiate a tear in the package 10. For example, the tear strength may be about 10-85% less than the tear strength of similar packaging without a discontinuous region 30. For example, if the tear strength of similar folded material without the discontinuous region is 0.10 N, then the tear strength of the inventive package would be about 0.010-0.085 N at a fold across the discontinuous region, where the tear is initiated at the discontinuous region. A 20% error tolerance is acceptable in measurements of this type of packaging. In an aspect of this invention, the tear strength of the inventive discontinuous region should be 25-75% less than the tear strength of similar materials without the discontinuous region. Preferably, the tear strength of the package is at least 50% less.

The embodiments of the present invention may include flexible packaging that affects and maintains an inert environment for a pharmaceutical product. For example, this protection includes protection from light, air, water, and other contaminants. Though the embodiments of the present invention provide protection from the ambient environment, the tearable package of the present invention yields easily along the predefined, discontinuous location such that users with limited grasp and grip abilities may open the package easily, while the design more difficultly accessed by children.

A number of embodiments of the invention have been described. Nevertheless, it will be understood that various modifications may be made without departing from the spirit and scope of the invention. Also, the features and elements described above may be modified in various ways than described above, where appropriate. Accordingly, alternative embodiments are within the scope of the following claims.

What is claimed is:

1. A tearable package for a pharmaceutical product comprising:
(i) a first layer comprising a sheet of flexible plastic and or foil
(ii) a second layer comprising a sheet of flexible plastic and or foil;
said first and second layers being overlaid with the other and fused together at a perimetrical sealing region, said fused layers defining a cavity therebetween for the storage of said pharmaceutical product;
wherein one of said layers has a discontinuous region in said sealing region, and the other layer has a continuous region covering the discontinuous region of the other layer; and
wherein said package may be folded on an axis through the discontinuous region, and when a tearing force is applied, said discontinuous region facilitates tear initiation along the fold axis through said layers and into the cavity of said package to allow the contents of the cavity to be removed.

2. The package of claim 1, further comprising an anchor portion opposite the discontinuous region, proximally located to the cavity so as to prevent a tear off portion from completely dissociating from the packaging.

3. The package of claim 1, where the discontinuous region is one selected from the group consisting of: a linear slit, a plurality of linear slits, a V shape, a W shape, a cross shape, a circle, a plurality of circles, a perforation, a plurality of perforations, and combinations thereof.

4. The package of claim 1, where the discontinuous region comprises one or more perforations through an entire sheet layer of the package.

5. The package of claim 1, where the sheet layers are of the same thicknesses.

6. The package of claim 1, where the sheet layers are different thicknesses.

7. The package of claim 1, further comprising a marking at the site of the discontinuous region on the sheet bearing the discontinuous region.

8. The package of claim 1, further wherein the marking is on the continuous layer opposite to the site of the discontinuous region for indicating the discontinuous region which is a tear origin.

9. The package of claim 1, wherein the package has a fold.

10. The package of claim 1, wherein the force required to tear the package open at the discontinuous region is about 10-85% of the force required at a position of the package without a discontinuous region.

11. The package of claim 1, wherein the force required to tear the package open at the discontinuous region is about 25-75% of the force required at a position of the package without a discontinuous region.

12. The package of claim 1, wherein the force required to tear the package open at the discontinuous region is about 50% of the force required at a position of the package without a discontinuous region.

13. The package of claim 1, further including a pharmaceutical product contained within the cavity.

14. The package of claim 1, wherein the pharmaceutical product is a film.

15. The package of claim 1, wherein the pharmaceutical product is a capsule or a tablet.

16. The package of claim 12, wherein the pharmaceutical product is a moistened towel or a pre-impregnated wipe.

17. A tearable package for a pharmaceutical product comprising:
(i) a first layer comprising a sheet of flexible plastic and or foil, said first layer having a first thickness;
(ii) a second layer comprising a sheet of flexible plastic and or foil, said second layer having a second thickness greater than said first thickness, said first and second layers being overlaid with the other and fused together at a perimetrical sealing region, said fused layers defining a cavity therebetween for the storage of said pharmaceutical product; and
wherein said package may be folded on an axis through the perimetrical sealing region, such that when a tearing force is applied, said different thicknesses facilitates tear initiation along the fold axis through said first layer and into the cavity of said package to allow the contents of the cavity to be removed.

18. The package of claim 17, further wherein the second layer remains integrally attached to the packaging upon tearing and accessing the cavity.

19. The package of claim 17, further comprising a discontinuous region configured to one of the layers in said sealing region, wherein the discontinuous region overlays upon a continuous region of the other layer to indicate and facilitate a tear region.

20. The package of claim 17, further wherein the cavity houses one selected from the group essentially of: a pharmaceutical film, a breath film, a cream; a liquid; a pill; a capsule, a moist towel; a pre-impregnated wipe, and combinations thereof.

21. A method for opening a package containing a pharmaceutical product, said method comprising:
a) providing a tearable package for a pharmaceutical product comprising:
(i) a first layer comprising a sheet of flexible plastic and or foil
(ii) a second layer comprising a sheet of flexible plastic and or foil;
said first and second layers being overlaid with the other and fused together at a perimetrical sealing region, said fused layers defining a cavity therebetween for the storage of said pharmaceutical product;
wherein one of said layers has a tear promoting feature in said sealing region;
b) folding one edge of the package on an axis that crosses the tear promoting feature and applying a tearing force to initiate a tear at the site of the tear promoting feature along the fold axis; and
c) propagating the tear through the layer defining the cavity to allow the contents of the cavity to be removed.

22. The method of claim 21, wherein the tearing force is applied by hand.
